(19) United States
(12) Patent Application Publication
(10) Pub. No.: US 2007/0043431 A1
(43) Pub. Date: Feb. 22, 2007

(54) Prosthetic valve

(75) Inventor: Jeffry S. Melsheimer, Springville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/506,459

(22) Filed: Aug. 17, 2006

Related U.S. Application Data
(60) Provisional application No. 60/709,956, filed on Aug. 19, 2005.

(51) Int. Cl. A61F 2/06

(52) U.S. Cl. 623/1.24; 600/36; 623/2.12

(57) ABSTRACT

Prosthetic valves and a method for making a prosthetic valve for implantation in a body site are provided. The prosthetic valve includes at least one flexible member movable between a first position that permits fluid flow in a first direction and a second position that substantially prevents fluid flow in a second direction. The flexible member has a proximal portion and a distal portion. The valve includes a receptacle operatively connected to the proximal portion of flexible member. The receptacle has an expanded position adapted to receive fluid flowing in the second direction and a contracted position adapted to allow fluid flow through the valve in the first direction. The valve further includes an attachment portion operably connected to the receptacle for attaching the valve to the body site.

### RELATED APPLICATIONS

This application claims the benefit of U.S. Provisional Application No. 60/709,956, filed Aug. 19, 2005, which is incorporated herein in its entirety.

### FIELD OF THE INVENTION

The present invention relates to medical devices, and in particular to prosthetic valve devices, methods of making such devices, and methods of deploying such devices within a body site.

### BACKGROUND

Many vessels in animals transport fluids from one bodily location to another. Frequently, fluid flows in a substantially unidirectional manner along the length of the vessel. For example, veins in the body transport blood to the heart and arteries carry blood away from the heart.

In mammalian veins, natural valves are positioned along the length of the vessel in the form of leaflets disposed annularly along the inside wall of the vein which open to permit blood flow toward the heart and close to restrict back flow. These natural venous valves open to permit the flow of fluid in the desired direction, and close upon a change in pressure, such as a transition from systole to diastole. When blood flows through the vein, the pressure forces the valve leaflets apart as they flex in the direction of blood flow and move towards the inside wall of the vessel, creating an opening therebetween for blood flow. When the pressure differential across the valve, the flow velocity, or both change, the leaflets return to a closed position to restrict or prevent blood flow in the opposite, i.e. retrograde, direction. The leaflet structures, when functioning properly, extend radially inwardly toward one another such that the tips contact each other to restrict backflow of blood.

In the condition of venous insufficiency, the valve leaflets do not function properly. Incompetent venous valves can result in symptoms such as swelling and varicose veins, causing great discomfort and pain to the patient. If left untreated, venous insufficiency can result in excessive retrograde blood flow through incompetent venous valves, which can cause venous stasis ulcers of the skin.

There generally are two types of venous insufficiency: primary and secondary. Primary venous insufficiency typically occurs where the valve structure remains intact, but the vein is simply too large in relation to the leaflets so that the leaflets cannot come into adequate contact to prevent backflow. More common is secondary venous insufficiency, where the valve structure is damaged, for example, by clots which gel and scar, thereby changing the configuration of the leaflets, i.e. thickening the leaflets and creating a “stub-like” configuration. Venous insufficiency can occur in the superficial venous system, such as the saphenous veins in the leg, or in the deep venous system, such as the femoral and popliteal veins extending along the back of the knee to the groin.

A common method of treatment of venous insufficiency is placement of an elastic stocking around the patient's leg to apply external pressure to the vein. Although sometimes successful, the tight stocking is quite uncomfortable, especially in warm weather, as the stocking must be constantly worn to keep the leaflets in apposition. The elastic stocking also affects the patient's physical appearance, thereby potentially having an adverse psychological affect. This physical and/or psychological discomfort can lead to the patient removing the stocking, thereby preventing adequate treatment.

Surgical methods for treatment of venous insufficiency have also been developed. A vein with incompetent venous valves can be surgically constricted to bring incompetent leaflets into closer proximity in an attempt to restore natural valve function. Methods for surgical constriction of an incompetent vein include implanting a frame around the outside of the vessel, placing a constricting suture around the vessel, or other types of treatment of the outside of the vessel to induce vessel contraction. Other surgical venous insufficiency treatment methods include bypassing or replacing damaged venous valves with autologous sections of veins with competent valves. However, these surgeries often result in a long patient recovery time and scarring, and carry the risks, e.g. anesthesia, inherent with surgery.

Recently, various implantable prosthetic devices and minimally invasive methods for implantation of these devices have been developed to treat venous insufficiency, without the disadvantages of treatment with an outer stocking or surgery. Such prosthetic venous valve devices can be inserted intravascularly, for example from an implantation catheter. Prosthetic devices can function as a replacement valve, or restore native valve function by bringing incompetent valve leaflets into closer proximity.

It is desirable to have prosthetic valve devices for implantation in a body site having at least one member for permitting fluid flow in a first direction and substantially preventing fluid flow in a second direction and having a receptacle for receiving fluid in the second flow direction as taught herein, methods of making such devices, and methods of deploying such devices in a body vessel. It is also desirable to have prosthetic valve devices having folded configurations to form portions of the valve device thereby reducing the number of seals, either by mechanical means or adhesives, to form the valve device and methods for forming such folded configurations.

### BRIEF SUMMARY

In one aspect of the present invention, a prosthetic valve for implantation in a body site is provided. The prosthetic valve includes at least one flexible member movable between a first position that permits fluid flow in a first direction and a second position that substantially prevents fluid flow in a second direction. The flexible member has a proximal portion and a distal portion. The valve includes a receptacle operatively connected to the proximal portion of flexible member. The receptacle has an expanded position adapted to receive fluid flowing in the second direction and a contracted position adapted to allow fluid flow through the valve in the first direction. The valve further includes an attachment portion operably connected to the receptacle for attaching the valve to the body site.

In another aspect of the present invention, a prosthetic valve for implantation into a body site is provided. The valve includes a flexible member and a receptacle together movable between an open configuration permitting fluid flow in a first direction and a closed configuration substantially preventing fluid flow in a second direction. The valve further includes an attachment portion operably connected to the receptacle for attaching the valve to a body site. The flexible member and the receptacle comprise a biocompatible material and are integrally formed.

In another aspect of the present invention, a method of making a prosthetic valve device for implantation in a body site is provided. The method includes forming a flexible member, the flexible member being movable between a first position that permits fluid flow in a first direction and a second position that substantially prevents fluid flow in a second direction. The method further includes forming a receptacle having an expended position for receiving fluid flow in the second direction and a contracted position for allowing fluid flow in a first direction through an opening in the valve. The method also includes providing an attachment portion operably connected to the receptacle for implanting the valve in the body vessel and assembling the valve for implantation into the body vessel.

Advantages of the present invention will become more apparent to those skilled in the art from the following description of the preferred embodiments of the invention which have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1A is a perspective view of an embodiment of the present invention in a vessel in a closed configuration;

FIG. 1B is a perspective view of the embodiment shown in FIG. 1 in a open configuration;

FIG. 2A is a perspective view of an embodiment of the present invention having a pair of leaflets shown in the closed configuration;

FIG. 2B is a perspective view of the embodiment shown in FIG. 2A in the open configuration;

FIG. 3A is a perspective view of an embodiment of the present invention having a single leaflet shown in the closed configuration;

FIG. 3B is a perspective view of the embodiment shown in FIG. 3A in the open configuration;

FIG. 4A is a front view of a leaflet and receptacle of the present invention;

FIG. 4B is a front view of an alternative shape of the embodiment shown in FIG. 4A;

FIG. 4C is a front view of an alternative shape of the embodiment shown in FIG. 4A;

FIG. 5A is a side view of a leaflet and receptacle an embodiment of the present invention;

FIG. 5B is a side view of an alternative shape of the embodiment shown in FIG. 5A;

FIG. 6 is a perspective view of an embodiment of the present invention having a frame;

FIG. 7 is a partial perspective view of an alternative embodiment of the present invention having a woven portion;

FIG. 8A is a top view of a square sheet for forming a prosthetic valve;

FIG. 8B is a top view of the square shown in FIG. 8A showing a first fold;

FIG. 8C is a top view of the square shown in FIG. 8A showing fold lines;

FIG. 8D is a top view of the square shown in FIG. 8C with the corners folded in;

FIG. 8E is a top view of the sheet shown in FIG. 8D with further folds;

FIG. 8F is a bottom view of the sheet shown in FIG. 8E;

FIG. 8G is a top view of the sheet shown in FIG. 8F folded in half to form a rectangle; and

FIG. 8H is a perspective view of an embodiment formed by folding in a closed configuration.

### DETAILED DESCRIPTION OF THE INVENTION

The present invention relates to medical devices, and in particular to prosthetic valves having at least one leaflet and a receptacle connected thereto for implantation in a body site for regulation of fluid flow through the body site. The valves of the present invention are suitable for implantation into ducts, canals, and other passageways in the body, as well as cavities and other locations. For example, the valves of the present invention are suitable for implantation into the vessels of the vasculature, such as veins, for regulating fluid flow through the vessel. The valves of the present invention may also be implanted in a passageway of the heart to regulate the fluid flow into and out of the heart.

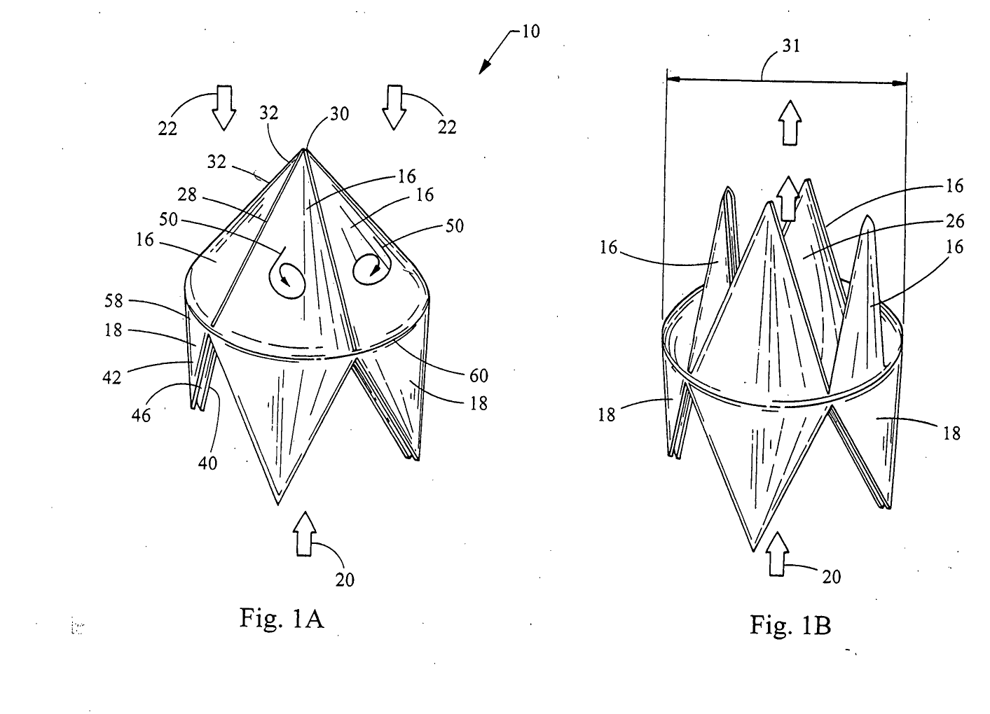

An embodiment of a prosthetic valve device 10 of the present invention is shown in FIG. 1A and described with respect to implantation into a vessel wall. The term “implantation” as used herein refers to the positioning of a valve device of the present invention in a particular environment, either temporarily, semi-permanently, or permanently. Permanent fixation of the valve device in a particular position is not required.

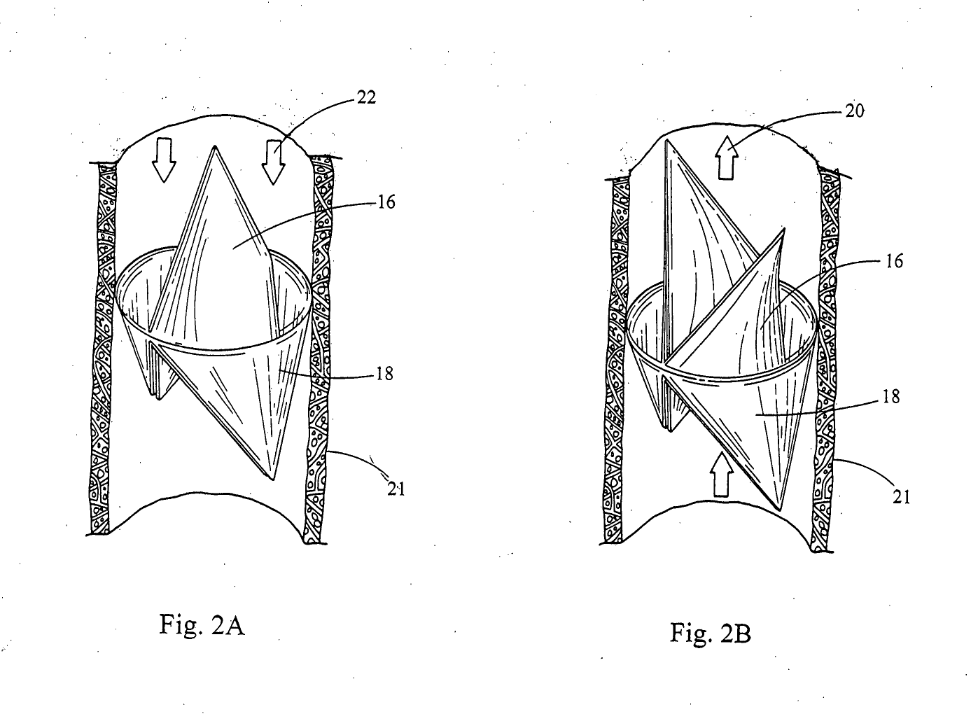

The valve device 10, as shown in FIG. 1A, includes a plurality of leaflets 16, each leaflet 16 being connected to a receptacle 18. The leaflets 16 and receptacles 18 may be formed with a flexible material and move in response to fluid flow in a first direction 20, i.e. towards the heart, and in a second, generally opposite direction 22. Preferably, a portion of the valve device 10, such as the leaflets 16 and the receptacles 18 may be formed by folding a sheet of material as described below in Example 1 or by molding the valve device 10 on a mandrel as described in Example 2. The valve device 10 may include may include one leaflet 16 and receptacle 18, or a plurality of leaflets 16 and receptacles 18, e.g. two, three, four, five or more leaflets, within the scope of the present invention. Fluid flow in the second direction 22 is shown in FIG. 1A where the leaflets 16 move inward toward the center of a vessel 21 (shown in FIG. 2A, for example) to meet each other at a contact region 28 formed between two leaflets 16 to close an opening 26 through the valve device 10. The receptacles 18 also move in response to the fluid flow as discussed below. When the fluid flow is in the first direction 20, the leaflets 16 move toward the wall of the vessel 21 to facilitate fluid flow through the opening 26.

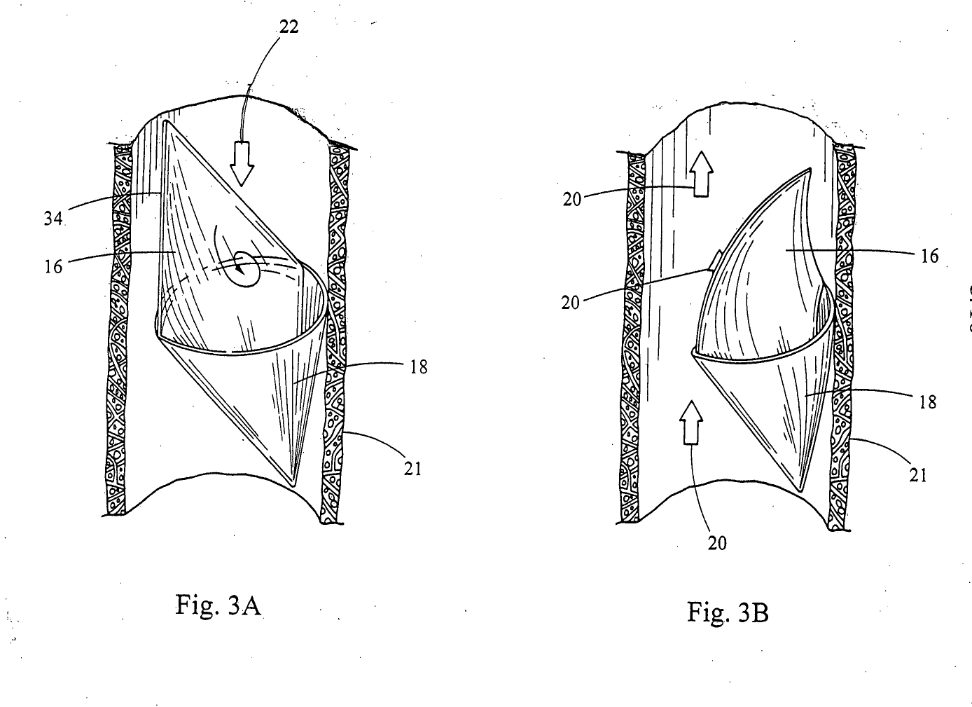

The leaflets 16 contact each other at the leaflet contact region 28 at a distal portion 30 of the valve device 10 when fluid flow is in the second direction 22. As shown in FIG. 1, four leaflets 16 meet together having the contact region 28 formed between two adjacent leaflets 16 along side portions 32 of the leaflet 16. The leaflet contact region 28 may also be formed at the distal portion 30 where all the leaflets 16 meet. When the valve 10 comprises two leaflets 16, the leaflet contact region 28 may be formed at the distal portion 30 as shown in FIG. 2A. When the valve 10 comprises a single leaflet 16, a vessel contact surface 34 may be formed at the distal portion 30 and the single leaflet 16 and receptacle 18 may be dimensioned and attached to the vessel 21 to allow the leaflet 16 together with the receptacle 18 to extend across the entire lumen of the vessel 21 as shown in FIG. 3A.

Each receptacle 18 is operatively connected to each leaflet 16 and moves in response to fluid flow in the first direction 20 and the second direction 22. As shown in FIG. 1A, the receptacles 18 extend proximally from the leaflets 16 and expand to form a conically shaped pocket for receiving fluid when the flow is in the second direction 22. Each receptacle 18 includes an inner wall 40 generally toward the center of the vessel 21 and outer wall 42 generally toward the wall of the vessel 21. The inner wall 40 and the outer wall 42 are joined together at a perimeter 46 to form the receptacle 18 for receiving fluid in the second direction 22. Preferably, the receptacles 18 are dimensioned to create flow vortices 50 similar to flow vortices formed in native valves that help to prevent fluid from pooling or stagnating in the receptacles 18. When the fluid flow is in the first direction 20, as shown in FIG. 1B, the receptacles 18 together with the leaflets 16 move toward the wall of the vessel 21 as fluid flows through the opening 26. The receptacles 18 collapse as the inner walls 40 and the outer walls 42 move closer together and toward the wall of the vessel 21. Fluid present in the receptacles 18 when the fluid flow is in the second direction 22 gets pushed out of the receptacles 18 as the inner walls 40 and the outer walls 42 move together.

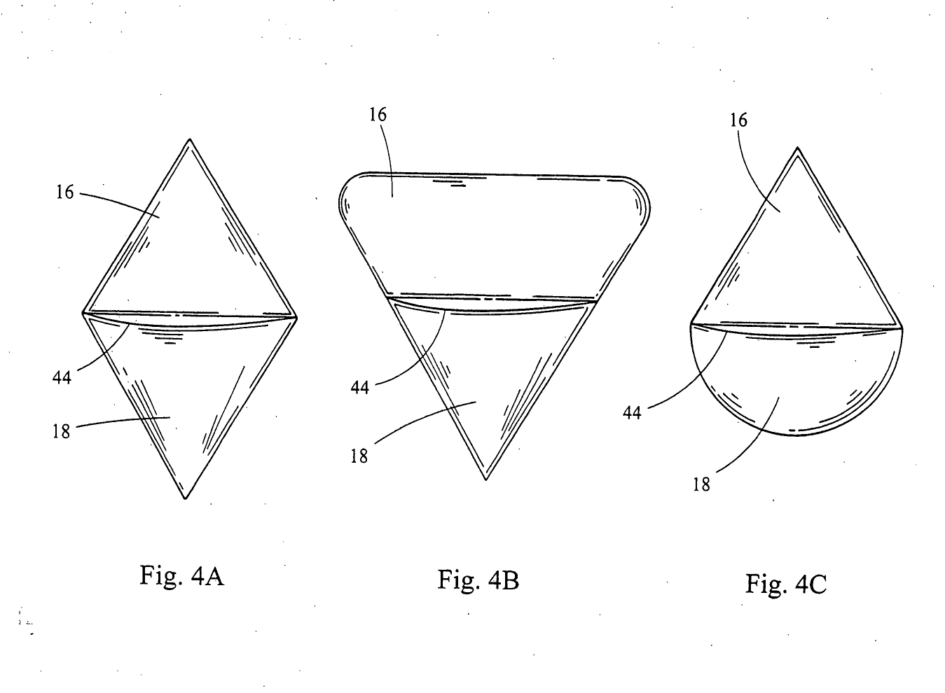

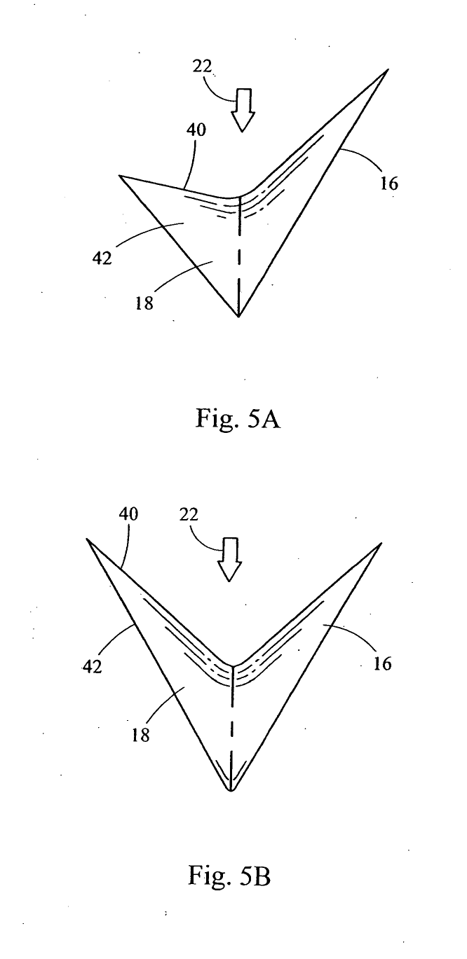

The shape of the leaflet 16 and the receptacle 18 will vary depending on the number of leaflets 16 and receptacles 18 and the body site for implantation and the like. One of skill in the art will recognize that the leaflets 16 may have any shape suitable for forming a contact with other leaflets 16 or the vessel wall 21 to allow flow in the first direction 20 and substantially prevent fluid flow in the second direction 22. The receptacles 18 may have any shape suitable for expanding and receiving fluid in the second direction 22 and for collapsing when fluid flow is in the first direction 20. As shown in FIGS. 4A-C, the shape of the leaflets 16 and chambers 18 may be triangular, curvilinear, or any shape that will allow the leaflets 16 to form a contact region and the receptacle 18 to form a pocket for receiving fluid in the second direction 22. The shape of the leaflet 16 may be the same as the shape of the receptacle 18 or different. For example, as shown in FIG. 1A, the leaflets 16 may be triangular to facilitate the meeting of four leaflets 16 together at the distal portion 30 of the valve device 10 and the receptacles 18 may also be triangular. As shown in FIGS. 5A and 5B, the receptacles 18 may have the outer wall 42 that extends distally a partial length compared to the length of the leaflet 16 (FIG. 5A) or the outer wall 42 may extend the full length compared to the length of the leaflet 16 (FIG. 5B).

Each leaflet 16 is operably connected to the receptacle 18 as discussed above. The leaflet 16 and the receptacle 18 may be integrally connected, formed by unitary construction from the same material as discussed below. Alternatively, the leaflet 16 may be formed separately from the receptacle 18 and operably connected after formation at a connection area 44 (shown in FIGS. 4A-4C). The leaflets 16 may have the same flexibility as the receptacles 18 or the relative flexibility of the leaflets 16 and the receptacles 18 may be different, for example, the leaflets 16 may be stiffer than the receptacles 18. The leaflets 16 themselves may have differing flexibility, for example, alternating between more flexible and less flexible when a plurality of leaflets are included in the valve device 10. The leaflet 16 and the receptacle 18 may be connected by any method know to one of skill in the art, including but not limited to, a hinge, sutures, staples, screws, rivets, and adhesives. Preferably, the connection area 44 between the leaflet 16 and the leaflet 18 will allow flexible movement of the valve device 10 in response to fluid flow in the first direction 20 and the second direction 22 and the connection are 44 will not interfere with the contacting of the leaflets 16 at the leaflet contact region 28.

The leaflet contact region 28 comprises a longitudinal portion along the valve device 10 in which the adjacent surfaces of leaflets 16 coapt or lie in close proximity to one another. Preferably, the leaflets 16 may be shaped and sized to provide a sufficient leaflet contact region 28 to decrease the amount of retrograde flow in the second direction 22 through the opening 26 formed between the leaflets 16 compared to the fluid flow in the first direction. One of skill in the art will understand how to maximize the leaflet contact region 28, for example, by lengthening the leaflets 16 longitudinally along the longitudinal axis of the vessel 21 with respect to the diameter of the vessel 21 into which the valve device 10 is implanted. Preferably, by lengthening the leaflet contact region 28, the valve device 10 will substantially seal during retrograde flow in the direction 22 so that undesired retrograde flow through the opening 26 may be minimized.

The size of the valve device 10 will depend on the size of the body site into which the valve device 10 will be implanted. Generally, the valve device for implantation into a vessel wall will range from about 5 mm to about 35 mm, although other sizes are possible. The expanse of the leaflets 16 at the opening 26 will vary depending on the size of the valve device 10 as well as the length of the leaflet contact region 28. In an average sized valve device 10 having a length of 25 mm, the preferred range of the coaptable leaflet contact region 28 may comprise 10-80% of the valve device 10 length (2.5-20 mm). A more preferred leaflet contact area 28 may comprise 30-60%, with 35-55% being most preferred. The relationship between leaflet contact area 28 and the diameter of the vessel 21 may be a factor in optimizing the functionality of the valve device 10. Preferably, the length of the leaflet contact region 28 is 25-250% of the nominal vessel diameter 31, with a more preferred range of 25-150%.

Preferably, but not essentially, the valve device 10 is configured such that the distance formed between the leaflets 16 in their fully open position, for example, shown in FIG. 1B, and the vessel diameter 31 remains preferably between 0-100% of the vessel diameter 31, with a more preferred range of 20-80% of the vessel diameter 31, and a most preferred range of 50-70% of the vessel diameter 31. In addition, the amount of slack in the leaflet 16 material also helps to determine how well the leaflets 16 coapt during retrograde flow in the second direction 22 and how large the opening 26 the leaflets 16 permit during flow in the first direction 20. Preferably, the leaflets 16 may be sized and shaped so that regular contact the outer walls of the vessel 21 may be diminished, especially when the leaflets 16 are formed from a bioremodelable material, such as an ECM, which can partially adhere to the wall of the vessel 21 over time as tissue grows into the leaflets 16 thus compromising the functionality of the valve device 10.

An edge portion 58 of the valve device 10 may further include adaptations for attachment to the vessel wall 21. As shown in FIG. 1A, the edge portion 58 may include an attachment area 60 having plurality of elements configured to partially or completely penetrate the body vessel walls, for example barbs or hooks (not shown). The adaptations for attachment to the vessel wall 21 may be provided on a portion of the attachment area 60 or the adaptations may be provided on the entire periphery defined by the attachment area 60. For example, barbs may be individually secured to the valve device 10 or barbs may be provided along a wire element. The wire element itself does not constitute a stent, as the wire element itself does not serve to exert radial force upon the vessel wall to retain the position of the device as would a stent. As will be understood by one of skill in the art, the number and location of adaptations on the attachment area 60 will be sufficient to secure the valve device 10 to the vessel 21 temporarily, semi-permanently or permanently. Exemplary attachment methods and devices are described in WO 2004/089253, which is herein incorporated in its entirety.

Alternatively or in addition, the attachment area 60 may be provided with a biocompatible adhesive or sealant sufficient to secure the edge portion 58 of the valve device 10 to the vessel wall 21. Any biocompatible adhesive known to one of skill in the art may be used. Nonlimiting examples of sealants and adhesives suitable for use with the valve device of the present invention include FOCALSEAL® (biodegradable eosin-PEG-lactide hydrogel requiring photopolymerization with Xenon light wand) produced by Focal; BERIPLAST® produced by Adventis-Bering; VIVOSTAT® produced by ConvaTec (Bristol-Meyers-Squibb); SEALAGEN™ produced by Baxter; FIBRX® (containing virally inactivated human fibrinogen and inhibited-human thrombin) produced by CryoLife; TISSEEL® (fibrin glue composed of plasma derivatives from the last stages in the natural coagulation pathway where soluble fibrinogen is converted into a solid fibrin) and TISSUCOL® produced by Baxter; QUIXIL® (Biological Active Component and Thrombin) produced by Omrix Biopharm; a PEG-collagen conjugate produced by Cohesion (Collagen); HYSTOACRYL® BLUE (ENBUCRILATE) (cyanoacrylate) produced by Davis & Geck; NEXACRYL™ (N-butyl cyanoacrylate), NEXABOND™, NEXABOND™ S/C, and TRAUMASEAL™ (product based on cyanoacrylate) produced by Closure Medical (TriPoint Medical); DERMABOND™ which consists of 2-Octyl Cyanoacrylate produced by Dermabond (Ethicon); TISSUEGLU® produced by Medi-West Pharma; and VETBOND™ which consists of n-butyl cyanoacrylate produced by 3 M. Additional adhesives and sealants known to one of skill in the art may be used with the valve device 10.

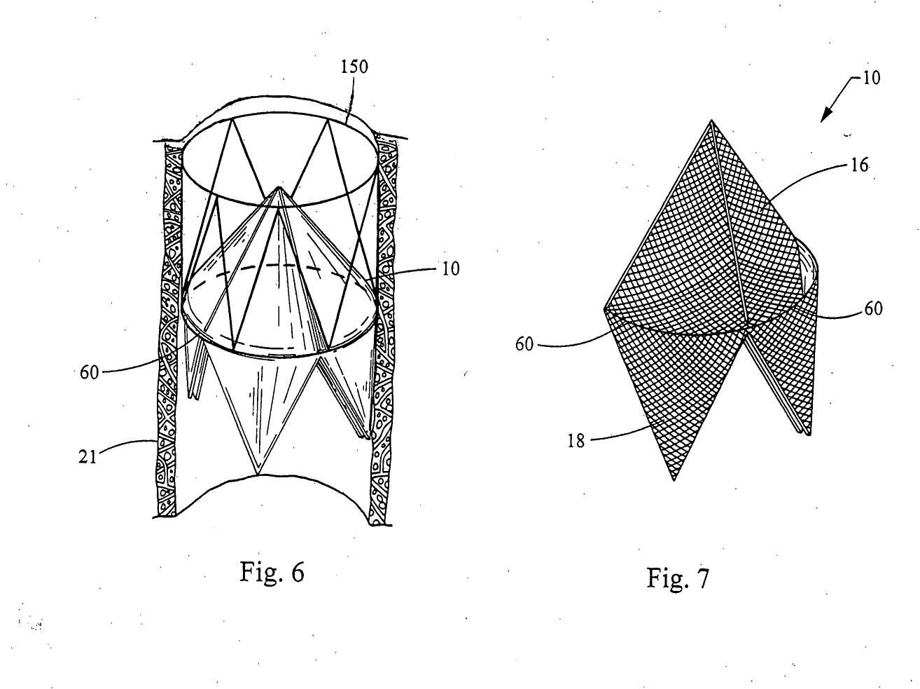

In some embodiments of the present invention, the attachment area 60 may further include a support frame 150 for further support and implantation of the valve device 10. As shown in FIG. 6, the frame 150 extends from the attachment area 60 and contacts the wall of the vessel 21.

Any suitable support frame can be used as the support frame 150 in the valve device 10. The specific support frame chosen will depend on several considerations, including the size and configuration of the vessel at the implantation site and the size and nature of the valve device 10.

A support frame that provides a stenting function, i.e., exerts a radially outward force on the interior of the body vessel in which the valve device 10 is implanted, may be used if desired. Numerous examples of support frames acceptable for use with the valve device 10 exist in the art and any suitable stent can be used as the support frame 150. Exemplary configurations for the support frame 150 include, but are not limited to, braided strands, helically wound strands, ring members, consecutively attached ring members, tube members, and frames cut from solid tubes. If a stent is used as the support frame 150, the specific stent chosen will depend on several factors, including the vessel into which the valve device is being implanted, the axial length of the treatment site, the number of valves desired in the device, the inner diameter of the body vessel, the delivery method for placing the support frame, and others. Those skilled in the art can determine an appropriate stent based on these and other factors.

The illustrated support frame 150 is an expandable support frame having radially compressed and radially expanded configurations, allowing the valve device 10 to be delivered to and implanted at a point of treatment using percutaneous techniques and devices. The support frame 150 can be either balloon- or self-expandable. In some embodiments, the self-expanding support frame 150 can be compressed into a low-profile delivery conformation and then constrained within a delivery system for delivery to a point of treatment in the lumen of a body vessel. At the point of treatment, the self-expanding support frame 150 can be released and allowed to subsequently expand to another configuration.

The support frame can have any suitable size. The exact configuration and size chosen will depend on several factors, including the desired delivery technique, the nature of the body vessel in which the valve device 10 will be implanted, and the size of the vessel. The support frame can be sized so that the second, expanded configuration is slightly larger in diameter that the inner diameter of the vessel in which the medical device will be implanted. This sizing can facilitate anchoring of the valve device 10 within the vessel wall 21 and maintenance of the valve device 10 at a point of treatment following implantation.

Examples of suitable frames 150 for use in the valve of the present invention include those described in U.S. Pat. Nos. 6,508,833; 6,464,720; 6,231,598; 6,299,635; 4,580,568; and U.S. Patent Application Publication Nos. 2004/018658 A1 and 2005/0228472 A1, all of which are hereby incorporated by reference in their entirety.

The valve 10 may further include one or more imageable materials located on the valve 10 that are configured to facilitate placement of the valve 10 in the vessel wall 21 in the desired orientation. The imageable materials may be viewed by devices such as a fluoroscope, X-ray, ultrasound, M.R.I., and others known to one of skill in the art. For example, radiopaque substances containing tantalum, barium, iodine, or bismuth, e.g. in powder form, can be coated upon or incorporated within the materials used to form the valve 10, such that, the location of the valve 10 is detectable. Exemplary prosthetic valve devices and imageable materials are further described in U.S. Publication No. 2004/0167619, which is incorporated by reference herein in its entirety.

The valve device 10 of the present invention may be delivered to a lumen of a body vessel by various techniques known in the art. By way of non-limiting example, the valve device 10 may be delivered and positioned in the body vessel using a catheter. For delivery, the valve device 10 may be placed in a folded or unexpanded configuration to fit in the lumen of a delivery catheter. The catheter is then introduced into the body vessel and its tip positioned at a point of treatment within the body vessel. The valve device 10 may then be expelled from the tip of the catheter at the point of treatment. Once expelled from the catheter, the valve device 10 may expand to the expanded configuration and engage the interior wall of the body vessel, preferably using attachment portion provided on the valve device. The valve device 10 may be self-expanding or expandable by a balloon of a balloon catheter as will be understood by one of skill in the art. Delivery has been described using a delivery catheter as an example, the valve device 10 may be delivered to a position within a body by any means known to one of skill in the art. Exemplary delivery devices suitable for implanting the valve 10 include U.S. Publication Nos. 2004/0225344 and 2003/0144670, which are incorporated by reference herein in their entirety.

Alternatively, rapid exchange catheters may be used, such as a rapid exchange delivery balloon catheter which allows exchange from a balloon angioplasty catheter to a delivery catheter without the need to replace the angioplasty catheter wire guide with an exchange-length wire guide before exchanging the catheters. Exemplary rapid exchange catheters that may be used to deliver the valve device of the present invention are described in U.S. Pat. Nos. 5,690,642; 5,814,061; and 6,371,961 which are herein incorporated by reference in their entirety.

Portions of the valve 10, including but not limited to, the leaflets 16, the receptacles 18, and the support frame 150 may be formed from a woven mesh. The mesh may include a bioabsorbable material, a synthetic material and combinations thereof (materials described below). For example, portions of the valve 10 may be formed by weaving a memory metal, such as NiTi with SIS or THORALON®. The weave may be uniform or non-uniform and have a single-ply or more than one ply. Extensions of a weave material, for example, a metal, may be used to form the attachment area 60 of the valve 10, a portion of the valve 10 formed by weaving and having extensions is shown in FIG. 7.

The valve device 10 may be made from a variety of materials known to one of skill in the art. The valve device 10 may be made from a single material or a combination of materials. The material or materials need only be biocompatible or able to be rendered biocompatible. The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The valve device 10 including, but not limited to, the leaflets 16, receptacles 18, the attachment area 60, and the support frame 150 may comprise a biocompatible material that can be degraded and absorbed by the body over time to advantageously eliminate the portion formed from the bioabsorbable material from the vessel before, during or after the remodeling process. Examples of suitable materials include natural materials, synthetic materials, and combinations of natural and synthetic materials. The biocompatible material may be, but is not required to be resorbable. As used herein, the term “resorbable” refers to the ability of a material to be absorbed into a tissue and/or body fluid upon contact with the tissue and/or body fluid. The contact can be prolonged, and can be intermittent. A number of resorbable materials are known in the art and any suitable material may be used. The material may also provide a matrix for the regrowth of autologous cells.

A number of bioabsorbable homopolymers, copolymers, or blends of bioabsorbable polymers are known in the medical arts. These include, but are not necessarily limited to, polyesters including poly-alpha hydroxy and poly-beta hydroxy polyesters, polycaprolactone, polyglycolic acid, polyether-esters, poly(p-dioxanone), polyoxaesters; polyphosphazenes; polyanhydrides; polycarbonates including polytrimethylene carbonate and poly(iminocarbonate); polyesteramides; polyurethanes; polyisocyantes; polyphosphazines; polyethers including polyglycols polyorthoesters; expoxy polymers including polyethylene oxide; polysaccharides including cellulose, chitin, dextran, starch, hydroxyethyl starch, polygluconate, hyaluronic acid; polyamides including polyamino acids, polyester-amides, polyglutamic acid, poly-lysine, gelatin, fibrin, fibrinogen, casein, and collagen.

Examples of biocompatible homo- or co-polymers suitable for use in the present invention include vinyl polymers including polyfumarate, polyvinylpyrolidone, polyvinyl alcohol, poly-N-(2-hydroxypropyl)-methacrylamide, polyacrylates, and polyalkylene oxalates.

Reconstituted or naturally-derived collagenous materials can be used in the present invention. Such materials that are at least bioresorbable will provide advantage in the present invention, with materials that are bioremodelable and promote cellular invasion and ingrowth providing particular advantage.

Suitable bioremodelable materials can be provided by collagenous extracellular matrix materials (ECMs) possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics or thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient.

Submucosa or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

For example, when a portion of the valve is formed from an ECM, such as small intestine submucosa (SIS), the SIS may be used in a sheet form as described above. SIS is commercially available from Cook Biotech, West Lafayette, Ind.

Portions of the valve device 10, including, but not limited to, the leaflets 16, the receptacles 18, the attachment area 60, and the support frame 150 may be formed from the same material or different materials. Examples of suitable materials for portions of the valve 10 include, without limitation, stainless steel (such as 316 stainless steel), nickel titanium (NiTi) alloys, e.g., Nitinol, other shape memory and/or superelastic materials, MP35N, gold, silver, a cobalt-chromium alloy, tantalum, platinum or platinum iridium, or other biocompatible metals and/or alloys such as carbon or carbon fiber, cellulose acetate, cellulose nitrate, silicone, cross-linked polyvinyl alcohol (PVA) hydrogel, cross-linked PVA hydrogel foam, polyurethane, polyamide, styrene isobutylene-styrene block copolymer (Kraton), polyethylene teraphthalate, polyurethane, polyamide, polyester, polyorthoester, polyanhidride, polyether sulfone, polycarbonate, polypropylene, high molecular weight polyethylene, polytetrafluoroethylene, or other biocompatible polymeric material, or mixture of copolymers thereof, or stainless steel, polymers, and any suitable composite material.

In some embodiments, the frame itself, or any portion of the frame, can be comprise one or more metallic bioabsorbable materials. Suitable metallic bioabsorbable materials include magnesium, titanium, zirconium, niobium, tantalum, zinc and silicon and mixtures and alloys. For example, a zinc-titanium alloy such as discussed in U.S. Pat. No. 6,287,332 to Bolz et al., which is incorporated herein by reference in its entirety, can be used. The metallic bioabsorbable material can further contain lithium, sodium, potassium, calcium, iron and manganese or mixtures thereof. For example, an alloy containing lithium:magnesium or sodium:magnesium can be used. The physical properties of the frame can be controlled by the selection of the metallic bioabsorbable material, or by forming alloys of two or more metallic bioabsorbable materials. For example, when 0.1% to 1%, percentage by weight, titanium is added to zinc, the brittle quality of crystalline zinc can be reduced. In another embodiment, when 0.1% to 2%, percentage by weight, gold is added to a zinc-titanium alloy, the grain size of the material is reduced upon curing and the tensile strength of the material increases.

In some embodiments of the present invention, at least a portion of the valve device 10 may be formed from biocompatible polyurethanes such as THORALON® (THORATEC, Pleasanton, Calif.). Portions of the valve device 10 include, but are not limited to, the leaflets 16, the receptacles 18, the attachment area 60 and the frame 150. The valves of the present invention or portions thereof may be formed with a variety of materials, including biocompatible polyurethanes. One example of a biocompatible polyurethane is THORALON (THORATEC, Pleasanton, Calif.). As described in U.S. Pat. Nos. 4,675,361 and 6,939,377, both of which are incorporated herein by reference. THORALON is a polyurethane base polymer blended (referred to as BPS-215) with a siloxane containing surface modifying additive (referred to as SMA-300). The concentration of the surface modifying additive may be in the range of 0.5% to 5% by weight of the base polymer.

The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

THORALON can be manipulated to provide either porous or non-porous THORALON. Porous THORALON can be formed by mixing the polyetherurethane urea (BPS-215), the surface modifying additive (SMA-300) and a particulate substance in a solvent. The particulate may be any of a variety of different particulates, pore forming agents or inorganic salts. Preferably the particulate is insoluble in the solvent. Examples of solvents include dimethyl formamide (DMF), tetrahydrofuran (THF), dimethyacetamide (DMAC), dimethyl sulfoxide (DMSO), or mixtures thereof. The composition can contain from about 5 wt % to about 40 wt % polymer, and different levels of polymer within the range can be used to fine tune the viscosity needed for a given process. The composition can contain less than 5 wt % polymer for some spray application embodiments. The particulates can be mixed into the composition. For example, the mixing can be performed with a spinning blade mixer for about an hour under ambient pressure and in a temperature range of about 18° C. to about 27° C. The entire composition can be cast as a sheet, or coated onto an article such as a mandrel or a mold. In one example, the composition can be dried to remove the solvent, and then the dried material can be soaked in distilled water to dissolve the particulates and leave pores in the material. In another example, the composition can be coagulated in a bath of distilled water. Since the polymer is insoluble in the water, it will rapidly solidify, trapping some or all of the particulates. The particulates can then dissolve from the polymer, leaving pores in the material. It may be desirable to use warm water f. or the extraction, for example water at a temperature of about 60° C. The resulting pore diameter can be substantially equal to the diameter of the salt grains.

The porous polymeric sheet can have a void-to-volume ratio from about 0.40 to about 0.90. Preferably the void-to-volume ratio is from about 0.65 to about 0.80. Void-to-volume ratio is defined as the volume of the pores divided by the total volume of the polymeric layer including the volume of the pores. The void-to-volume ratio can be measured using the protocol described in AAMI (Association for the Advancement of Medical Instrumentation) VP20-1994, Cardiovascular Implants—Vascular Prosthesis section 8.2.1.2, Method for Gravimetric Determination of Porosity. The pores in the polymer can have an average pore diameter from about 1 micron to about 400 microns. Preferably the average pore diameter is from about 1 micron to about 100 microns, and more preferably is from about 1 micron to about 10 microns. The average pore diameter is measured based on images from a scanning electron microscope (SEM). Formation of porous THORALON is described, for example, in U.S. Pat. No. 6,752,826 and U.S. Patent Application Publication No. 2003/0149471 A1, both of which are incorporated herein by reference.

Non-porous THORALON can be formed by mixing the polyetherurethane urea (BPS-215) and the surface modifying additive (SMA-300) in a solvent, such as dimethyl formamide (DMF), tetrahydrofuran (THF), dimethyacetamide (DMAC), dimethyl sulfoxide (DMSO). The composition can contain from about 5 wt % to about 40 wt % polymer, and different levels of polymer within the range can be used to fine tune the viscosity needed for a given process. The composition can contain less than 5 wt % polymer for some spray application embodiments. The entire composition can be cast as a sheet, or coated onto an article such as a mandrel or a mold. In one example, the composition can be dried to remove the solvent.

THORALON has been used in certain vascular applications and is characterized by thromboresistance, high tensile strength, low water absorption, low critical surface tension, and good flex life. THORALON is believed to be biostable and to be useful in vivo in long term blood contacting applications requiring biostability and leak resistance. Because of its flexibility, THORALON is useful in larger vessels, such as the abdominal aorta, where elasticity and compliance is beneficial.

A variety of other biocompatible polyurethanes may also be employed. These include polyurethane ureas that preferably include a soft segment and include a hard segment formed from a diisocyanate and diamine. For example, polyurethane ureas with soft segments such as polytetramethylene oxide, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

The diisocyanate used as a component of the hard segment may be represented by the formula OCN—R—NCO, where —R— may be aliphatic, aromatic, cycloaliphatic or a mixture of aliphatic and aromatic moieties. Examples of diisocyanates include tetramethylene diisocyanate, hexamethylene diisocyanate, trimethyhexamethylene diisocyanate, tetramethylxylylene diisocyanate, 4,4′-decyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-diisocyanate, 1-methoxyphenyl 2,4-diisocyanate, 4,4′-biphenylene diisocyanate, 3,3-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures.

Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, neopentyl alcohol, 1,6-hexanediol, 1,8-octanediol, propylene glycols, 2,3-butylene glycol, dipropylene glycol, dibutylene glycol, glycerol, or mixtures thereof.

Biocompatible polyurethanes modified with cationic, anionic and aliphatic side chains may also be used. See, for example, U.S. Pat. No. 5,017,664.

Other biocompatible polyurethanes include: segmented polyurethanes, such as BIOSPAN; polycarbonate urethanes, such as BIONATE; and polyetherurethanes such as ELASTHANE; (all available from POLYMER TECHNOLOGY GROUP, Berkeley, Calif.).

Other biocompatible polyurethanes include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10,-20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL -AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible polyurethanes may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

Additional examples of suitable materials for portions of the valve 10 include, without limitation, suitable metals or metal alloys include: stainless steels (e.g., 316, 316L or 304), nickel-titanium alloys including shape memory or superelastic types (e.g., nitinol or elastinite); inconel; noble metals including copper, silver, gold, platinum, paladium and iridium; refractory metals including molybdenum, tungsten, tantalum, titanium, rhenium, or niobium; stainless steels alloyed with noble and/or refractory metals; magnesium; amorphous metals; plastically deformable metals (e.g., tantalum); nickel-based alloys (e.g., including platinum, gold and/or tantalum alloys); iron-based alloys (e.g., including platinum, gold and/or tantalum alloys); cobalt-based alloys (e.g., including platinum, gold and/or tantalum alloys); cobalt-chrome alloys (e.g., elgiloy); cobalt-chromium-nickel alloys (e.g., phynox); alloys of cobalt, nickel, chromium and molybdenum (e.g., MP35N or MP20N); cobalt-chromium-vanadium alloys; cobalt-chromium-tungsten alloys; platinum-iridium alloys; platinum-tungsten alloys; magnesium alloys; titanium alloys (e.g., TiC, TiN); tantalum alloys (e.g., TaC, TaN); L605; magnetic ferrite; bioabsorbable materials, including magnesium; or other biocompatible metals and/or alloys thereof. Shape memory alloys are known in the art and are discussed in, for example, “Shape Memory Alloys,” Scientific American, 281: 74-82 (November 1979), incorporated herein by reference. Other shape memory materials may also be utilized, such as, but not limited to, irradiated memory polymers such as autocrosslinkable high density polyethylene (HDPEX).

Other suitable materials used in the valve 10 include carbon or carbon fiber; cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene, polytetrafluoroethylene, or another biocompatible polymeric material, or mixtures or copolymers of these; polylactic acid, polyglycolic acid or copolymers thereof, a polyanhydride, polycaprolactone, polyhydroxybutyrate valerate or another biodegradable polymer, or mixtures or copolymers of these; a protein, an extracellular matrix component, collagen, fibrin or another biologic agent; or a suitable mixture of any of these.

In some embodiments of the present invention, the valve 10 or portion thereof may include one or more bioactive agents. Bioactive agents can be included in any suitable part of the valve prosthesis, for example in the support frame and/or the valve leaflet. Selection of the type of bioactive agent, the portions of the valve prosthesis comprising the bioactive agent and the manner of attaching the bioactive agent to the valve prosthesis can be chosen to perform a desired therapeutic function upon implantation and, in particular, to achieve controlled release of the bioactive agent.

For example, a therapeutic bioactive agent can be combined with a biocompatible polyurethane, impregnated in an extracellular collagen matrix material, incorporated in the support structure or coated over any portion of the valve prosthesis. In one embodiment, the valve prosthesis can comprise one or more valve leaflets comprising a bioactive agent coated on the surface of the valve leaflet or impregnated in the valve leaflet. In another aspect, a bioactive material is combined with a biodegradable polymer to form a portion of the support structure.

A bioactive agent can be incorporated in or applied to portions of the valve prosthesis by any suitable method that permits controlled release of the bioactive agent material and the effectiveness thereof for an intended purpose upon implantation in the body vessel. Preferably, the bioactive agent is incorporated into the support frame or coated onto the support frame. The configuration of the bioactive agent on or in the valve prosthesis will depend in part on the desired rate of elution for the bioactive agent. Bioactive agents can be coated directly on the valve prosthesis surface or can be adhered to a valve prosthesis surface by means of a coating. For example, a bioactive agent can be blended with a polymer and spray or dip coated on the valve prosthesis surface. For example, a bioactive agent material can be posited on the surface of the valve prosthesis and a porous coating layer can be posited over the bioactive agent material. The bioactive agent material can diffuse through the porous coating layer. Multiple porous coating layers and or pore size can be used to control the rate of diffusion of the bioactive agent material. The coating layer can also be nonporous wherein the rate of diffusion of the bioactive agent material through the coating layer is controlled by the rate of dissolution of the bioactive agent material in the coating layer.

The bioactive agent material can also be dispersed throughout the coating layer, by for example, blending the bioactive agent with the polymer solution that forms the coating layer. If the coating layer is biostable, the bioactive agent can diffuse through the coating layer. If the coating layer is biodegradable, the bioactive agent is released upon erosion of the biodegradable coating layer.

Bioactive agents may be bonded to the coating layer directly via a covalent bond or via a linker molecule which covalently links the bioactive agent and the coating layer. Alternatively, the bioactive agent may be bound to the coating layer by ionic interactions including cationic polymer coatings with anionic functionality on bioactive agent, or alternatively anionic polymer coatings with cationic functionality on the bioactive agent. Hydrophobic interactions may also be used to bind the bioactive agent to a hydrophobic portion of the coating layer. The bioactive agent may be modified to include a hydrophobic moiety such as a carbon based moiety, silicon-carbon based moiety or other such hydrophobic moiety. Alternatively, the hydrogen bonding interactions may be used to bind the bioactive agent to the coating layer.

The bioactive agent can optionally be applied to or incorporated in any suitable portion of the valve prosthesis. The bioactive agent can be applied to or incorporated in the valve prosthesis, a polymer coating applied to the valve prosthesis, a material attached to the valve prosthesis or a material forming at least a portion of the valve prosthesis. The bioactive agent can be incorporated within the material forming the support frame, or within holes or wells formed in the surface of the support frame. The valve prosthesis can optionally comprise a coating layer containing the bioactive agent, or combinations of multiple coating layers configured to promote a desirable rate of elution of the bioactive from the valve prosthesis upon implantation within the body.

A coating layer comprising a bioactive agent can comprise a bioactive agent and a biostable polymer, a biodegradable polymer or any combination thereof. In one embodiment, the bioactive agent is blended with a biostable polymer to deposit the bioactive agent within the porous channels within the biostable polymer that permit elution of the bioactive agent from the valve prosthesis upon implantation. Alternatively, a blend of the bioactive and the bioabsorbable polymer can be incorporated within a biostable polymer matrix to permit dissolution of the bioabsorbable polymer through channels or pores in the biostable polymer matrix upon implantation in the body, accompanied by elution of the bioactive agent.

Multiple coating layers can be configured to provide a valve prosthesis with a desirable bioactive agent elution rate upon implantation. The valve prosthesis can comprise a diffusion layer positioned between a portion of the valve prosthesis that comprises a bioactive agent and the portion of the valve prosthesis contacting the body upon implantation. For example, the diffusion layer can be a porous layer positioned on top of a coating layer that comprises a bioactive agent. The diffusion layer can also be a porous layer positioned on top of a bioactive agent coated on or incorporated within a portion of the valve prosthesis.

A porous diffusion layer is preferably configured to permit diffusion of the bioactive agent from the valve prosthesis upon implantation within the body at a desirable elution rate. Prior to implantation in the body, the diffusion layer can be substantially free of the bioactive agent. Alternatively, the diffusion layer can comprise a bioactive agent within pores in the diffusion layer. Optionally, the diffusion layer can comprise a mixture of a biodegradable polymer and a bioactive positioned within pores of a biostable polymer of a diffusion layer. In another embodiment, the porous diffusion layer can comprise a mixture of a biodegradable polymer and a biostable polymer, configured to permit absorption of the biodegradable polymer upon implantation of the valve prosthesis to form one or more channels in the biostable polymer to permit an underlying bioactive agent to diffuse through the pores formed in the biostable polymer.

In one embodiment, the valve prosthesis is coated with a coating of between about 1 μm and 50 μm, or preferably between 3 μm and 30 μm, although any suitable thickness can be selected. The coating can comprise a bioactive material layer contacting a separate layer comprising a carrier, a bioactive material mixed with one or more carriers, or any combination thereof. The carrier can be biologically or chemically passive or active, but is preferably selected and configured to provide a desired rate of release of the bioactive material. In one embodiment, the carrier is a bioabsorbable material, and one preferred carrier is poly-L-lactic acid. U.S. Publication No. 2004/0034409A1, published Feb. 19, 2004, describes methods of coating a bioabsorbable metal support frame with bioabsorbable materials such as poly-L-lactic acid that are incorporated herein by reference.

Medical devices comprising an antithrombogenic bioactive material are particularly preferred for implantation in areas of the body that contact blood. An antithrombogenic bioactive material is any bioactive material that inhibits or prevents thrombus formation within a body vessel. The medical device can comprise any suitable antithrombogenic bioactive material. Types of antithrombotic bioactive materials include anticoagulants, antiplatelets, and fibrinolytics. Anticoagulants are bioactive materials which act on any of the factors, cofactors, activated factors, or activated cofactors in the biochemical cascade and inhibit the synthesis of fibrin. Antiplatelet bioactive materials inhibit the adhesion, activation, and aggregation of platelets, which are key components of thrombi and play an important role in thrombosis. Fibrinolytic bioactive materials enhance the fibrinolytic cascade or otherwise aid is dissolution of a thrombus.

Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin.

Further examples of antithrombotic bioactive materials include anticoagulants such as heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717; antiplatelets such as eftibatide, tirofiban, orbofiban, lotrafiban, abciximab, aspirin, ticlopidine, clopidogrel, cilostazol, dipyradimole, nitric oxide sources such as sodium nitroprussiate, nitroglycerin, S-nitroso and N-nitroso compounds; fibrinolytics such as alfimeprase, alteplase, anistreplase, reteplase, lanoteplase, monteplase, tenecteplase, urokinase, streptokinase, or phospholipid encapsulated microbubbles; and other bioactive materials such as endothelial progenitor cells or endothelial cells.

Other examples of bioactive coating compounds include antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as (GP) II_{b}/IIIₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives (cyclosporine, tacrolimus (FK-506), sirolimus (rapamycin), tacrolimus, everolimus, azathioprine, mycophenolate mofetil); angiogenic agents: vascular endothelial growth factor (VEGF), fibroblast growth factor (FGF); angiotensin receptor blockers; nitric oxide and nitric oxide donors; anti-sense oligionucleotides and combinations thereof, cell cycle inhibitors, mTOR inhibitors, and growth factor receptor signal transduction kinase inhibitors; retenoids; cyclin/CDK inhibitors; endothelial progenitor cells (EPC); angiopeptin; pimecrolimus; angiopeptin; HMG co-enzyme reductase inhibitors (statins); metalloproteinase inhibitors (batimastat); protease inhibitors; antibodies, such as EPC cell marker targets, CD34, CD133, and AC 133/CD133; Liposomal Biphosphate Compounds (BPs), Chlodronate, Alendronate, Oxygen Free Radical scavengers such as Tempamine and PEA/NO preserver compounds, and an inhibitor of matrix metalloproteinases, MMPI, such as Batimastat. Still other bioactive agents that can be incorporated in or coated on a frame include a PPAR α-agonist, a PPAR □agonist and RXR agonists, as disclosed in published U.S. Publication No. 2004/007329, published Apr. 15, 2004, and incorporated in its entirety herein by reference.

In some embodiments of the present invention, it may be preferable to treat at least a portion of the valve device 10 for the following non-limiting reasons, including, to minimize adherence of portions of the valve device 10 to itself or to portions of the vessel wall, to increase resistance to biodegradation, to decrease antigenicity and to regulate retraction during remodeling. For example, a portion of the valve device 10 may be treated with a crosslinking agent to at least partially crosslink the remodelable material. Cross-linking agents include glutaraldehyde, carbodiimide, and polyepoxy containing agents. Compared with other known methods, glutaraldehyde (GA) crosslinking of collagen provides materials with the highest degree of crosslinking. Glutaraldehyde is a five carbon aliphatic molecule with an aldehyde at each end of the chain rendering it bifunctional. The aldehyde is able to chemically interact with amino groups on collagen to form chemical bonds. This crosslinking agent is readily available, inexpensive, and forms aqueous solutions that can effectively crosslink tissue in a relatively short period. Using GA crosslinking, increased resistance to biodegradation and reduced antigenicity improved mechanical properties of collagen-based materials can be achieved.

Various types of crosslinking agents are known in the art and can be used such as ribose and other sugars, oxidative agents and dehydrothermal (DHT) methods. For instance, one crosslinking agent is 1-ethyl-3-(3-dimethylaminopropyl)carbodiimide hydrochloride (EDC). Alternatively, sulfo-N-hydroxysuccinimide is added to the EDC crosslinking agent as described by Staros, J. V., Biochem. 21, 3950-3955, 1982. Besides chemical crosslinking agents, the layers of materials, such as remodelable materials, used to form portions of the valve device 10 may be bonded together by other means such as those described above. Other methods of crosslinking remodelable materials of the invention are disclosed, for example, in U.S. Pat. No. 6,117,979 to Hendricks et al. For instance, crosslinking can also be accomplished with diisocyanates by bridging of amine groups on two adjacent polypeptide chains. Another method of crosslinking involves the formation of an acyl azide. The acyl azide method involves the activation of carboxyl groups in the polypeptide chain. The activated groups form crosslinks by reaction with collagen amine groups of another chain. Alternatively, a method has recently been developed that does not need an esterification step or the use of hydrazine. In this method, a carboxyl group is converted to an acyl azide group in one single step by reaction with diphenylphosphorylazide (DPPA). Also, water-soluble carbodiimides can be used to activate the free carboxyl groups of glutamic and aspartic acid moieties in collagen. Yet another crosslinking method uses epoxy compounds to crosslink collagen. See, for example, U.S. Pat. No. 4,806,595 to Noishiki et al. and U.S. Pat. No 5,080,670 to Imamura et al. One technique for regulating remodelable retraction includes layering remodelable materials or aligning collagen fibers in various ways in one or more layers of the remodelable material. In one embodiment, the method of U.S. Pat. No. 6,572,650 to Abraham et al. can be used to prepare layers of extracellular matrix remodelable material bonded together by dehydrating them while in wrapped arrangement on a sleeve-covered mandrel. While not wishing to be bound by theory, it is believed that dehydration brings the extracellular matrix components, such as collagen fibers, in the layers together when water is removed from the spaces between the fibers in the matrix.

Portions of the valve device 10 may be treated in other ways to desirably affect the remodelable retraction of the body vessel wall, such as the extent, rate or location of remodelable retraction. One skilled in the art can also refer to other resources to provide such alternative treatments for remodelable materials. For example, U.S. Patent Application 2003/0175410 A1 of Campbell et al., published Sep. 18, 2003 and incorporated herein by reference, provides a variety of other treatments for remodelable materials that can influence remodeling properties. Textbooks such as “Basic & Clinical Pharmacology,” 6th Ed., Bertram G. Katzung, Ed., Appleton & Lange (1995) and Joel G. Hardman et al., Eds., “Goodman & Gilman's The Pharmacological Basis of Therapeutics,” 9th Ed., McGraw-Hill (1996) also provide various compounds that can be incorporated in the remodelable material to influence the remodelable contraction process.

### EXAMPLE 1

### Formation of a Valve Device by Folding

In some embodiments of the present invention, the valve device 10 may be formed by folding, thereby reducing the number of seams that must be physically sealed. Many methods and designs for forming the valve device 10 may be used. By way of non-limiting example, the valve device 10 may preferably be formed by folding a sheet 200 of material to form the leaflets 16 and the receptacles 18 and the opening 26 therethrough. The sheet 200 for forming the valve device 10 may be made from any biocompatible material known to one skilled in the art, including the materials described above, such as, but not limited to SIS and THORALON®. The sheet 200 may be formed from multiple layers, including combinations of different materials or multiple layers of the same material where the sheets may be adhered together using the adhesives described above or mechanically adhered together, for example by sonic bonding. The sheet 200 may also be formed from woven materials as described above. Alternatively, the sheet 200 may be formed in a single layer having multiple thicknesses at predetermined areas, for example, a single layer having a thickened portion formed along the attachment area 60 or along the contact areas 28. The materials and thicknesses for the sheet 200 may be selected based on tensile strength and column strength, for example where thickened portions may provide column strength to resist buckling and tensile strength to resist tearing. Additional configurations for the sheet 200 are possible as will be understood by one skilled in the art.

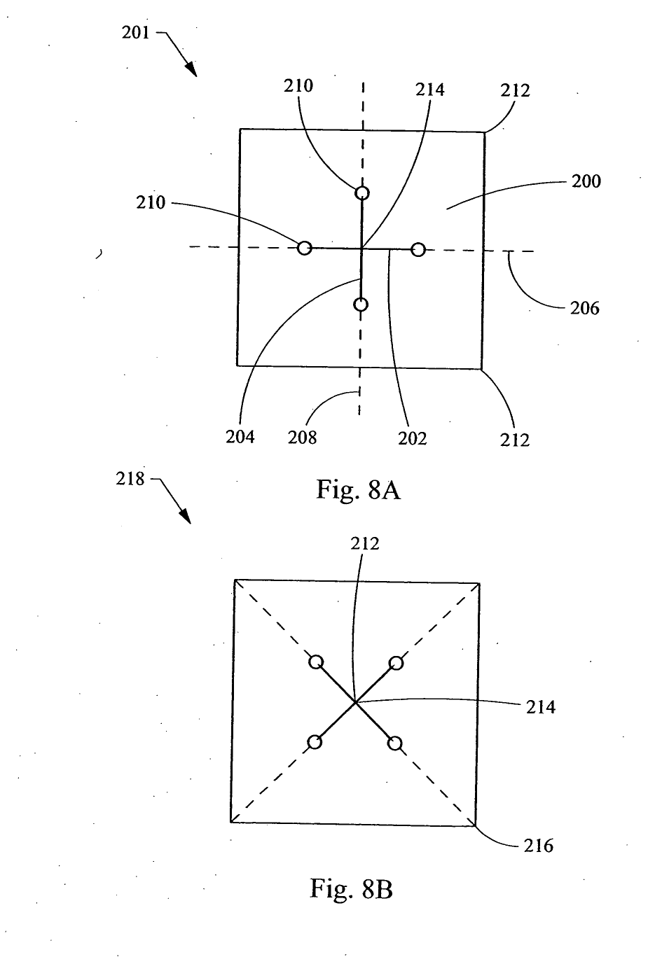

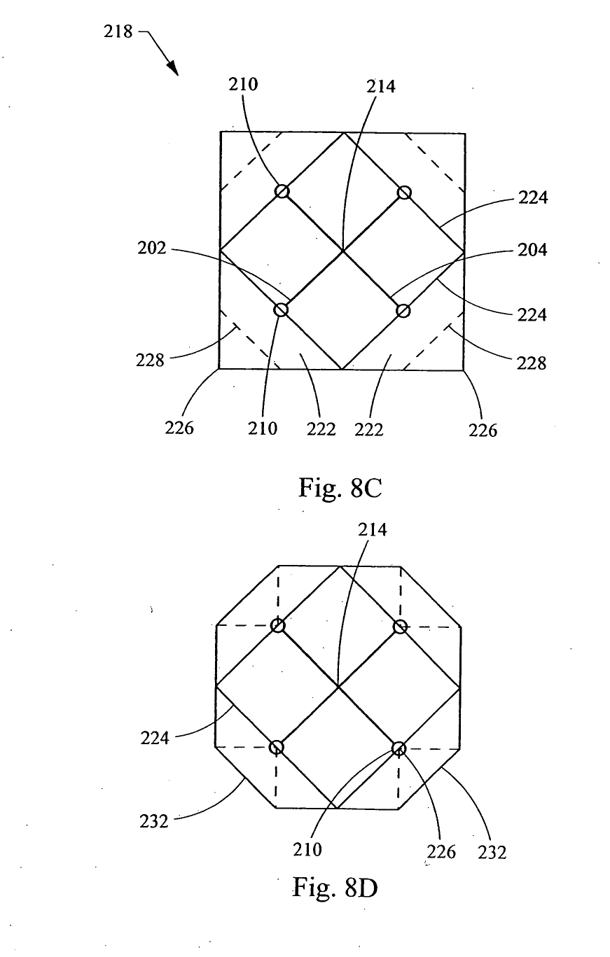

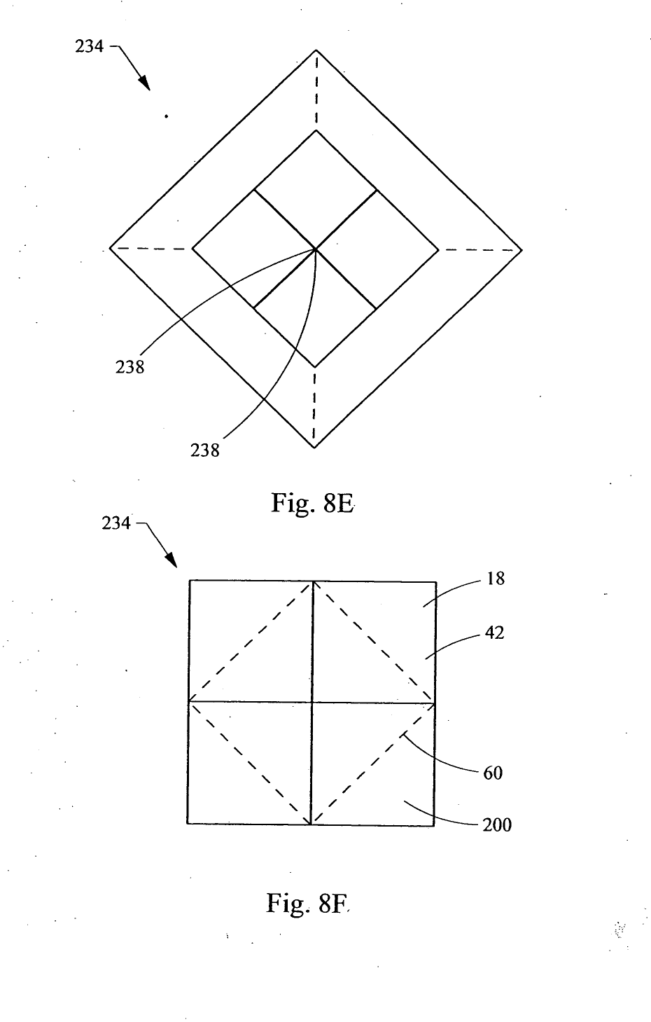

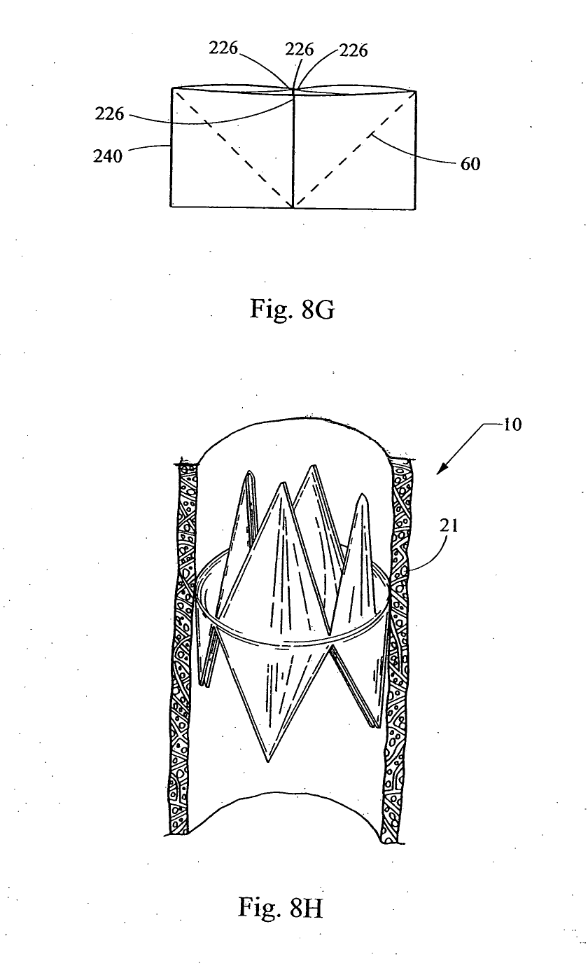

As shown in FIG. 8A, the sheet 200 may be in the shape of a square 201 for forming the valve device 10 by folding. A first opening 202 may be made in the center of the sheet 200 along a portion of a first axis 206 and a second opening 204 may be made, perpendicular to the first opening 202 and along a second axis 208, perpendicular to the first axis 206. Optionally, circular openings 210 may be made at the ends of the openings 202, 204 that will help with the formation of the valve device 10 by reducing overlapping material at the ends of the openings 202, 204.

Next, each one of four corners 212 is folded to the center 214 of the sheet 200 so that each corner 212 is at the center 214 and a second, smaller square 218 is formed from the sheet 200, shown in FIG. 8B. The sheet 200 is flipped and each corner 216 is folded into the center 214 to form a third, smaller square (not shown). This square is reopened to form the second square 218. As shown in FIG. 8C, triangles 222 formed by folds 224 from folding the sheet 200 to form the third square, are folded again by folding a corner 226 into the opening 210 at the line 228, shown in FIG. 8D. Next, edges 232 are folded at fold 224 toward the center 214, to form a fourth square 234, shown in FIG. 8E. FIG. 8F shows the opposite side of the square 234 showing the portion that forms the outer wall 42 of the receptacle 18 when the valve 10 formed from sheet 200 is expanded. The dashed lines show where the attachment area 60 is formed. The square 234 is folded in half to from a rectangle, unfolded and then refolded in half in the opposite direction to form a second rectangle 240 shown in FIG. 8G. The four corners 226 are expanded away from the rectangle 240 forming four closed pockets 238.

The four closed pockets 238 are then pulled together and directed downward to form the valve device 10. The corners 226 are folded downward and in half into the pocket 238 to form the valve device 10 shown in FIG. 8H. As described above and shown in FIGS. 5A and 5B, the outer wall 42 may be folded to form a partial wall or left unfolded to form the outer wall 42 having the same length as the inner wall 40.

### EXAMPLE 2

### Forming a Valve Device using a Mandrel

The valve device 10 may be formed from THORALON® using a mandrel to shape molten material in the form of a valve device. For example, the valve device 10 shown in FIGS. 1A and 1B having four leaflets 16 and receptacles 18 may be formed by using a triangular shaped, four-pronged, mandrel to form the receptacles 18 and leaflets 16 extending therefrom, with plates in between the four prongs to separate each of the receptacles 18 and to form the opening 26 in the valve device 10. The edge portion 58 may be formed on the mandrel being connected between each of the receptacles 18. Preparation of THORALON® for use with a mandrel, porous or nonporous, is described above.

Although the invention herein has been described in connection with a preferred embodiment thereof, it will be appreciated by those skilled in the art that additions, modifications, substitutions, and deletions not specifically described may be made without departing from the spirit and scope of the invention as defined in the appended claims. The scope of the invention is defined by the appended claims, and all devices that come within the meaning of the claims, either literally or by equivalence, are intended to be embraced therein.

1. A prosthetic valve for implantation in a body site, the valve comprising:
at least one flexible member movable between a first position that permits fluid flow in a first direction and a second position that substantially prevents fluid flow in a second direction, the at least one flexible member having a proximal portion and a distal portion; and
a receptacle operatively connected to the proximal portion of the at least one flexible member, the receptacle having an expanded position adapted to receive fluid flowing in the second direction and a contracted position adapted to allow fluid flow through the valve in the first direction, and
an attachment portion operably connected to the receptacle for attaching the valve to the body site.

2. The prosthetic valve of claim 1, wherein the valve comprises a plurality of flexible members, each of the flexible members having a receptacle operatively connected thereto.

3. The prosthetic valve of claim 1 wherein the valve comprises at least one biocompatible synthetic material.

4. The prosthetic valve device of claim 3, wherein the at least one biocompatible synthetic material is a polymeric material.

5. The prosthetic valve of claim 1, wherein the valve comprises a bioabsorbable material.

6. The prosthetic valve of claim 5, wherein the bioabsorbable material is small intestine submucosa.

7. The prosthetic valve of claim 1, wherein the attachment portion comprises a frame.

8. The prosthetic valve of claim 7 wherein the frame comprises a material selected from the group consisting of stainless steel, nickel, silver, platinum, gold, titanium, tantalum, iridium, tungsten, a self-expanding nickel titanium alloy and inconel.

9. The prosthetic valve of claim 1, wherein at least a portion of the valve comprises an antithrombogenic bioactive agent.

10. The prosthetic valve of claim 1 wherein at least a portion of the prosthetic valve comprises a plurality of layers of biocompatible materials.

11. The prosthetic valve device of claim 1 wherein at least a portion of the valve comprises a woven material.

12. A prosthetic valve for implantation in a body site, the valve comprising:
a flexible member and a receptacle together moveable between an open configuration permitting fluid flow in a first direction and a closed configuration substantially preventing fluid flow in a second direction; and
an attachment portion operably connected to the receptacle for attaching the valve to the body site;
wherein the flexible member and the receptacle comprise a biocompatible material and are integrally formed.

13. The prosthetic valve of claim 12 wherein the biocompatible material comprises a polyurethane.

14. The prosthetic valve of claim 12 wherein the biocompatible material comprises an extracellular matrix.

15. The prosthetic valve of claim 12 wherein the attachment portion comprises a frame.

16. A method of making a prosthetic valve device for implantation in a body site, the method comprising:
forming a flexible member, the flexible member being movable between a first position that permits fluid flow in a first direction and a second position that substantially prevents fluid flow in a second direction;
forming a receptacle having an expanded position for receiving fluid flow in the second direction and a contracted position for allowing fluid flow in the first direction through an opening in the valve;
providing an attachment portion operably connected to the receptacle for implanting the valve in the body site; and
assembling the valve for implantation into the body site.

17. The method of claim 16, comprising forming the flexible member and the receptacle by folding a sheet of material.

18. The method of claim 16 comprising forming the flexible member and the receptacle from a polymeric material.

19. The method of claim 16 comprising forming the flexible member and the receptacle from a bioabsorbable material.

20. The method of claim 16, comprising forming the flexible member and the receptacle using a mandrel.
